(19) United States
(12) Patent Application Publication
(10) Pub. No.: US 2020/0316868 A1
(43) Pub. Date: Oct. 8, 2020

(54) ULTRA-HIGH RESOLUTION 3D PRINTED ANATOMICAL AND STRUCTURAL MODELS

(71) Applicant: The Regents of the University of Colorado, a body corporate, Denver, CO (US)

(72) Inventor: Nicholas Jacobson, Denver, CO (US)

(21) Appl. No.: 16/375,132

(22) Filed: Apr. 4, 2019

(51) Int. Cl. B29C 64/393; G06T 17/05; G06F 17/50; G06F 9/30; G06T 19/20; A61B 5/00

(52) U.S. Cl. CPC B29C 64/393; G06T 17/05; A61B 5/0035; G06F 9/3004; G06T 19/20; G06F 17/50

(57) ABSTRACT

Systems and methods are provided for ultra-high resolution 3D printing anatomical and structural models. Some embodiments use a method of 3D volumetric printing that includes receiving imaging data (e.g., medical imaging data). Using the imaging data, a computer-aided design (CAD) model of the structure can be created. For example, in some embodiments the CAD model may be a voxel-based model or a stereolithography (STL) model. The CAD model can then be sliced into multiple horizontal layers to create a set of color files in a format supporting transparency. Using the set of color files, instructions can be generated to control a 3D printer supporting multiple materials to produce a 3D model of the structure using the multiple materials. In some embodiments, the 3D model of the structure produced by the 3D printer includes at least one layer with a gradient with two or more of the multiple materials.

### TECHNICAL FIELD

Various embodiments of the present technology generally relate to three-dimensional (3D) printing. More specifically, the embodiments of the present technology relate ultra-high resolution 3D printed anatomical and structural models (e.g., from DICOM files and digital structural analysis).

### BACKGROUND

Three-dimensional (3D) printing uses model from a digital file to create a physical 3D object. The 3D object is typically created by adding successive layers of material (e.g., plastics, metals, etc.) representing a thinly sliced horizontal cross-section of the model until the physical 3D object is created. The 3D printing process is sometimes referred to as additive manufacturing as each layer is added one on top of the other. Since the 3D printing process is creating the physical 3D object one layer at a time, complex functional objects can be created without the use of molds, milling, or other traditional subtractive manufacturing techniques. Applications span a variety of manufacturers and applications from simply rapid prototyping to consumer, medical, and automotive products.

3D printers can range from single material printers to printers that utilize multiple materials to build the physical 3D object. Different printers may use different technologies to build the physical 3D object. For example, some printers may melt or soften materials which are laid one on top of the other while other printers may use an ultra-violate light to cure layers containing a photo-reactive resin.

### SUMMARY

Systems and methods are described for ultra-high resolution 3D printed anatomical and structural models (e.g., from DICOM files and digital structural analysis). Some embodiments use a method of three-dimensional volumetric printing that includes receiving imaging data (e.g., medical imaging data) regarding an structure (e.g., an anatomical structure such as the heart). Using the imaging data, a computer-aided design (CAD) model of the structure can be created. For example, in some embodiments the CAD model may be a voxel-based model or a stereolithography (STL) model. The CAD model can then be sliced into multiple horizontal layers to create a set (or stack) of color files in a format supporting transparency (e.g., a GIF file format, a PNG file format, a BMP file format, a TIFF file format, or a JPEG 2000 file format). Using the set of color files representing the multiple layers of the CAD model, instructions can be generated to control a three-dimensional (3D) printer supporting multiple materials to produce a 3D model of the structure using the multiple materials. In some embodiments, the 3D model of the structure produced by the 3D printer includes at least one layer with a gradient with two or more of the multiple materials.

In some embodiments, the CAD model can be sliced into multiple layers to create the set of color files by first selecting an orientation of the CAD model. A viewpoint into a segment of the CAD model in the selected orientation can then be generated and the slice of the CAD model can be created and saved. This can be repeated until all segments of the CAD model have been captured. Some embodiments may post-process the set of color files based on printing parameters of the 3D printer. For example, in some embodiments, the printer parameters may be automatically identified (e.g., by querying the 3D printer or accessing a file with the printer parameters). The printer parameters can include but are not limited to the dimensions of a printing bed, x-resolution, and y-resolution. Using the printer parameters, the multiple layers within the set of color files can be scaled to allow the 3D model of the structure produced by the 3D printer to fit on the dimensions of the printing bed. In some embodiments, the post-processing of the set of color files can include quantizing and dither the multiple layers within the set of color files to correspond to the multiple materials available for printing via the 3D printer.

Embodiments of the present technology also include computer-readable storage media containing sets of instructions to cause one or more processors to perform the methods, variations of the methods, and other operations described herein.

Some embodiments provide for a system that includes a processor, a database, a 3D modeling module, a slicing module, a printing module, a 3D printer, and/or other components. The database can store multiple digital files representing a structure. The 3D modeling module can be configured to process the multiple digital files and create three-dimensional (3D) model of the structure from the multiple digital files. The slicing module can be configured to create multiple color files each representing a layer (e.g., a horizontal layer) of the structure. The multiple color files can support transparency to allow removal of one or more components of the structure without additional processing. The printing module can be configured to generate, based on the multiple color files representing the multiple layers of the structure, instructions configured to control a 3D printer supporting multiple materials to produce a physical 3D model of the structure using the multiple materials.

While multiple embodiments are disclosed, still other embodiments of the present technology will become apparent to those skilled in the art from the following detailed description, which shows and describes illustrative embodiments of the technology. As will be realized, the technology is capable of modifications in various aspects, all without departing from the scope of the present technology. Accordingly, the drawings and detailed description are to be regarded as illustrative in nature and not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present technology will be described and explained through the use of the accompanying drawings in which:

FIG. 1 illustrates an example of a communications environment in which some embodiments of the present technology may be utilized;

FIG. 2 illustrates an operational workflow according to one or more embodiments of the present technology;

FIG. 3 illustrates a set of components of a 3D printing system according to one or more embodiments of the present technology;

FIG. 4 is a flowchart illustrating a set of operations for generating an anatomical structure according to one or more embodiments of the present technology;

FIG. 5 is a flowchart illustrating a set of operations for slicing a model in accordance with some embodiments of the present technology;

FIG. 6 is flowchart illustrating a set of operations for post-processing sliced files in accordance with one or more embodiments of the present technology;

FIG. 7 is a flowchart illustrating a set of operations for generating a printable file by establishing a voxel grid and dithering the model in accordance with some embodiments of the present technology;

FIG. 8 is an example of a graphical user interface that may be used according to various embodiments of the present technology;

FIG. 9 illustrates an example of a 3D printed heart printed via the voxel-based 3D printing techniques according to one or more embodiments of the present technology;

FIG. 10 illustrates an example of a selected portion of a heart printed via the voxel-based 3D printing techniques in accordance with various embodiments of the present technology;

FIGS. 11-13 illustrate examples of a brain printed via the voxel-based 3D printing techniques in accordance with various embodiments of the present technology; and

FIG. 14 illustrates an example of a computer systemization that may be used in some embodiments of the present technology.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Various embodiments of the present technology generally relate to three-dimensional (3D) printing. More specifically, the embodiments of the present technology relate ultra-high resolution 3D printed anatomical and structural models (e.g., from DICOM files and digital structural analysis). A voxel is like a three-dimensional pixel. Much like a pixel, which describes the attributes of an element within a larger composition; a voxel can describe attributes about a physical location within a 3D volume. These attributes can include information about its material properties, density, color, and more.

Most, if not all, commercially available modeling software is built upon surface boundary representation paradigm, where a solid object is represented by its surface. Such software is unable to manage spatial variations in material properties, furthermore these models are limited in complexity due to file size limitations of computers and 3D printers. However, voxels offer a new paradigm where objects can be defined as a dense representation of material properties throughout a 3D volume with very small file sizes.

Leveraging voxel-based control of multi-material 3D printing, various embodiments of the present technology enable additive manufacturing of discontinuous data types (e.g., medical data) such as image and volumetric based data. As a result, some embodiments alleviate the need to postprocess data sets to boundary representations, preventing alteration of data and loss of information in the produced physicalizations.

Various embodiments of the present technology provide for a wide range of technical effects, advantages, and/or improvements to 3D printers, computing systems and components. For example, various embodiments include one or more of the following technical effects, advantages, and/or improvements: 1) use of a multiple images (e.g., 2D medical images) to create a 3D model capable of voxel-based 3D printing; 2) integrated use of computational design and availability of multiple materials voxel-based 3D printing; 3) use of unconventional and non-routine computer operations to enable 3D printing of multiple materials with gradients of the materials in the 3D printed structure; 4) use of unconventional and non-routine computer operations to allow selection and printing of areas of interest of the 3D model without post-processing; 5) removes limit on file size constraints by sequentially feeding layer instructions to the 3D printer; 6) use of unconventional and non-routine computer operations to generate complex 3D volumetric models; 7) allows for generation of printing instructions up to the printing resolution limits of any 3D printer; 8) use of accurate 3D printed physical structures from a variety of medical imaging data for presurgical planning with patient specific models with custom fit capabilities; and/or 9) unique manufacturing processes to create complex structures using droplet-based 3D printing instead of traditional continuous surface based printing.

In the following description, for the purposes of explanation, numerous specific details are set forth in order to provide a thorough understanding of embodiments of the present technology. It will be apparent, however, to one skilled in the art that embodiments of the present technology may be practiced without some of these specific details. While, for convenience, embodiments of the present technology are described with reference to printing of anatomical structures (e.g., for presurgical planning) by use of medical imaging and voxel-based 3D printing, embodiments of the present technology are equally applicable to analysis and printing of other structures.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

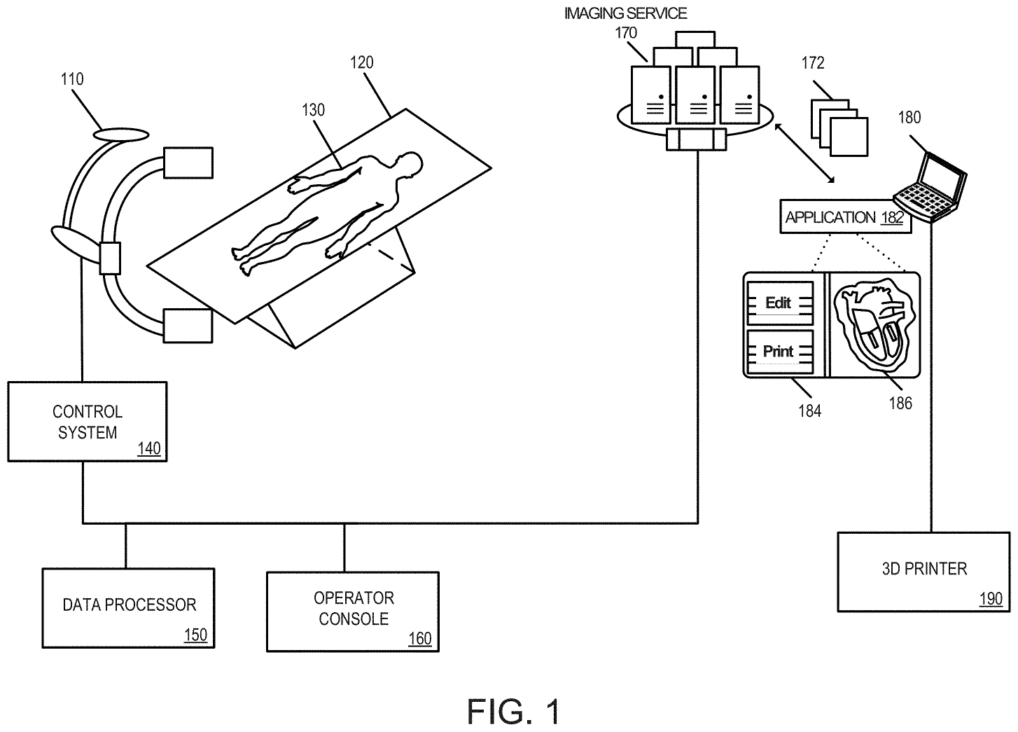

FIG. 1 illustrates an example of an operating environment 100 in which some embodiments of the present technology may be utilized. In the embodiments illustrated in FIG. 1, operating environment 100 may include imaging device 110, bed 120 to support patient 130, control system 140, data processor 150, operator console 160, imaging service 170, computing device 180, and 3D printer 190. Once patient 130 has been positioned and secured on bed 120, an operator can use operator console 160 to command the bed to move the patient to a desired location before activating imaging device 110. The desired scanning sequences can then be initiated to generate images and flows (e.g., 4D flows) of various body parts (e.g., heart, brain, lungs, wrists, knees, ankles, cartilage, etc.) of patient 130. The set of images and flows can be displayed on a screen or monitor associated with operator console 160 and stored within imaging service 170.

The operator can use operator console 160 to select and control the scans as well as review results. As the scan is selected by the operator, control system 140 controls imaging device 110 to scan patient 130. As the results from the scans are received, data processor 150 can process the data to generate one or more images that can be displayed via operator console 160. For example, data processor 150 may include one or more modules to transform a set of images into a motion model that can be displayed in operator console 160. The data returned from imaging device 110 and/or images created by data processor 150 can be stored using imaging service 170.

Control system 140 may be used in one or more embodiments of the present technology. Control system 140 can include a communication interface for communicating with imaging device 110 and operator console 160. In accordance with various embodiments, control system 140 can receive operator commands from operator console 160, process those requests, and issue commands to imaging device 110 indicating the scan sequence. Control system 140 may also include a physiological acquisition controller (not shown) that can receive signals from different sensors to identify gating or other physiological data. This information can detect additional patient information (e.g., movement, heart rate, respiratory patterns, etc.) which can be used in creating enhanced images by data processor 150. In some embodiments, imaging service 170 may receive the images in real-time or near real-time and evaluate the imaging data to identify whether the data is sufficient to create a 3D model of the structure. When imaging service 170 determines additional views or angles are needed, imaging service 170 can notify the operator and/or automatically schedule the additional scans of the patient.

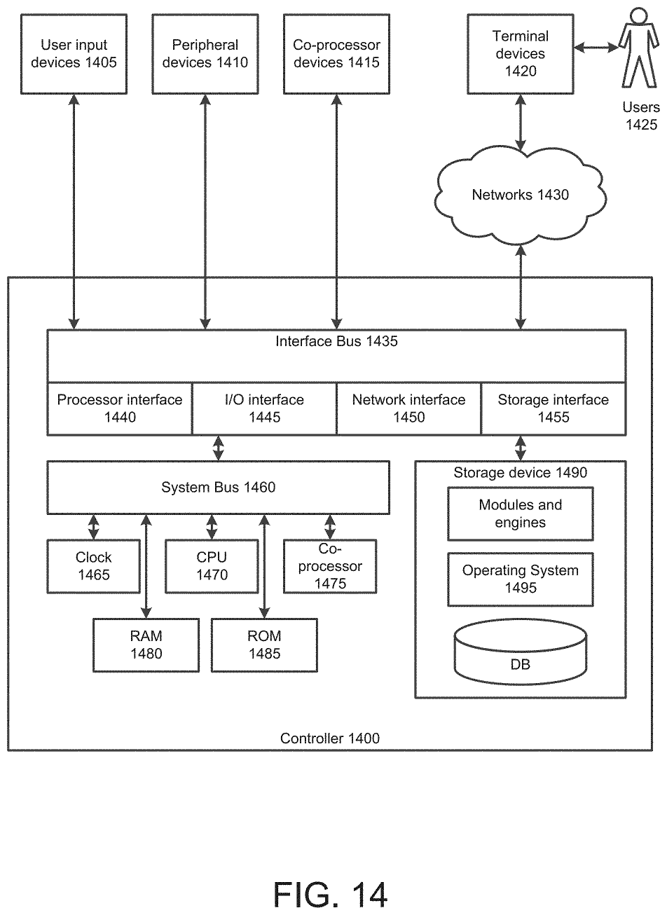

The images, collected data, and generated data (e.g., stress analysis, topological optimization, etc.) can be stored on imaging service 170. Files 172 may be stored in a variety of digital file formats (e.g., DICOM) which can be accessed by computing device 180 and used by application 182 (e.g., having interface portion 184 and visualization portion 186) for the generation of a 3D model which can be printed on 3D printer 190. Imaging service 170 is representative of any system or collection of systems that is configured to facilitate scheduling of scans for one or more imaging devices 110 identifying specific structures. Imaging service 170 can include server computers, blade servers, rack servers, and any other type of computing system (or collection thereof) suitable for employing the file storage and computational design for Such systems may employ one or more virtual machines, containers, or any other type of virtual computing resource in the context of identifying and replacing a least compatible object in a viewpoint with at least one more compatible object/product of which FIG. 14 is representative.

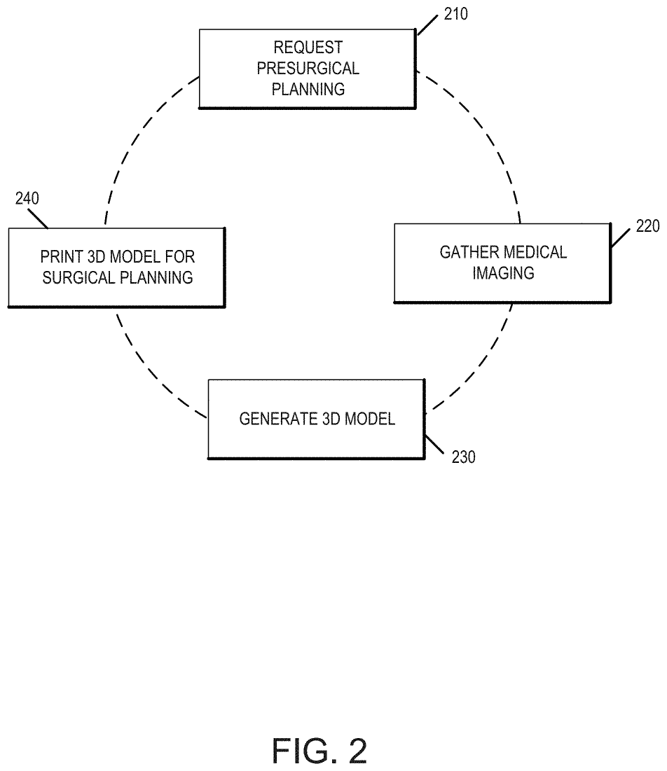

FIG. 2 illustrates an operational workflow 200 according to one or more embodiments of the present technology. As illustrated in FIG. 2, a user may submit a request for presurgical planning using request operation 210. The request may be submitted to a cloud-based analysis and 3D printing platform using a graphical user interface. Gathering operation 220 gather medical imaging data for the anatomical structure of interest. In accordance with various embodiments, the user may submit the images and data directly or may authorize a request for access to a particular set of data at an institution. Once the data has been collected, a 3D model of the anatomical structure can be created during generation operation 230.

In accordance with various embodiments, a voxel-based model or an STL model may be created. A voxel represents a data point (e.g., opacity, color, etc.) on a three-dimensional grid space representing the volume of a small portion of the structure. A voxel-based model does not explicitly encode the position of the voxel along with the assigned value. Instead, positions of the voxels are inferred based upon their relative positions to other voxels (e.g., a voxel's position in a data structure that makes up a single volumetric image). As a result, the size of the data structure needed to represent the anatomical model is much smaller than encoding schemes which would use explicitly representations identifying coordinates of vertices. Polygons represented by the STL model can efficiently represent simple 3D structures with lots of empty or homogeneously filled space, while voxel-based model can represent regularly sampled spaces that are non-homogeneously filled in an efficient manner. As such the type of computer-aided design model selected may depend on the properties of the scanned structure.

The 3D model created during generation operation 230 can then be sliced and possibly processed to create a set of instructions for printing the physical 3D model during printing operation 240. Various embodiments may use different payment models for the 3D printing. For example, in some embodiments the imaging service may assign a billing and/or insurance code to the model creation. This information can be fed to billing service were internal departments and/or external entities are charged for the printing and analysis.

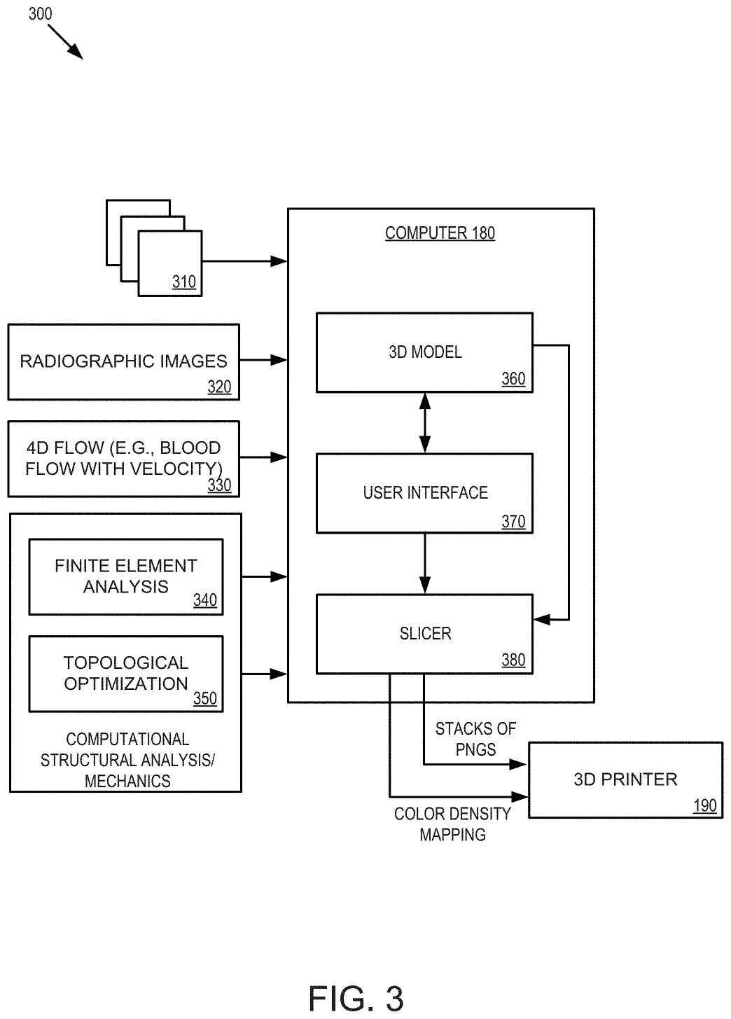

FIG. 3 illustrates a set of components of a 3D printing system 300 according to one or more embodiments of the present technology. As shown in FIG. 3, computing device 180 can receive a stack of images 310, radiographic images 320, 4D flow (e.g., with blood flow and velocity, and/or a variety of computational and structural analysis results (e.g., finite element analysis results 340, topological optimization (TO) results, and the like). For example, TO is a mathematical technique optimizing the material layout within a given design space, for a given set of loads, boundary conditions and constraints with the goal of maximizing the performance (e.g., maximizing stiffness) of the system. In accordance with various embodiments, TO integrated with 3D printing creates a new concept for embedding structure within a monolithic mass of material. In some embodiments, 4D flow allows doctors to understand how to better design interventions that might cause aneurysm or weakening of the veins.

While not illustrated in FIG. 3, computing device 180 can include various components such as a memory (e.g., volatile memory and/or nonvolatile memory), power supply (e.g., battery), processor(s) (e.g., application processor, graphical processors, coprocessors, etc.) for executing processing instructions and making computations, and an operating system. Additional components such as a data storage component (e.g., hard drive, flash memory, memory card, etc.), one or more network interfaces (e.g., Bluetooth Interface, and Network Communication Interface may be used to enable computing device 180 to communicate by transmitting and receiving wireless signals using licensed, semi-licensed or unlicensed spectrum over a telecommunications network), an audio interface, a microphone, a display, a keypad, and/or other input and/or output interfaces.

Once the data files are accessed, computer 180 can generate a 3D computer aided design (CAD) model 360 from the data files. In accordance with various embodiments, 3D model 360 can be a voxel-based model, an STL model, and/or other model that does not specifically encode the position of the voxel or polygonal shapes within the file. One benefit of using this type of model structure is that it allows for complex models to be created more efficiently and stored in smaller model sized. In addition, this file type also allows for the finer control of material at the volumetric level, rather than simply specifying the boundary surface. For example, 1 cubic inch of material contains 64,800,000,000 voxels that can be individually programmed and blended together in various embodiments to create functional gradients and a growing range of color combination due to the ability to blend materials at this level.

This model can be presented to the user via user interface 370 to allow for changes to the model 360, removal/addition of various structures, selection of areas of interest, and the like. Once 3D model 360 is finalized, the user can request 3D printing. The request is submitted to slicer 380 which accesses the 3D model 360 and generates a set of sliced horizontal images. In some embodiments, the sliced horizontal images may be a stack of color files that have a transparency property. Transparency allows for void in the printing process, where various embodiments of the system specify where no material is to be placed. In addition, transparency allows for dither to occur only between colored areas specified in the images. As such, the use of color files with transparency property allows for greater control.

This stack of color files can be transmitted (e.g., sequentially or in batches) to the 3D printer along with a color density mapping. In accordance with various embodiments, the color density mapping registers the colors to available materials in the 3D printer. Some embodiments can specify (e.g., in a text file) which material is to relate to which color. The material can be either color based or durometer based allowing for mechanical properties. When combining color with mechanical properties, various embodiments of the present technology can create a full color structure (e.g. a heart) with the same mechanical properties of the actual structure (e.g., the heart).

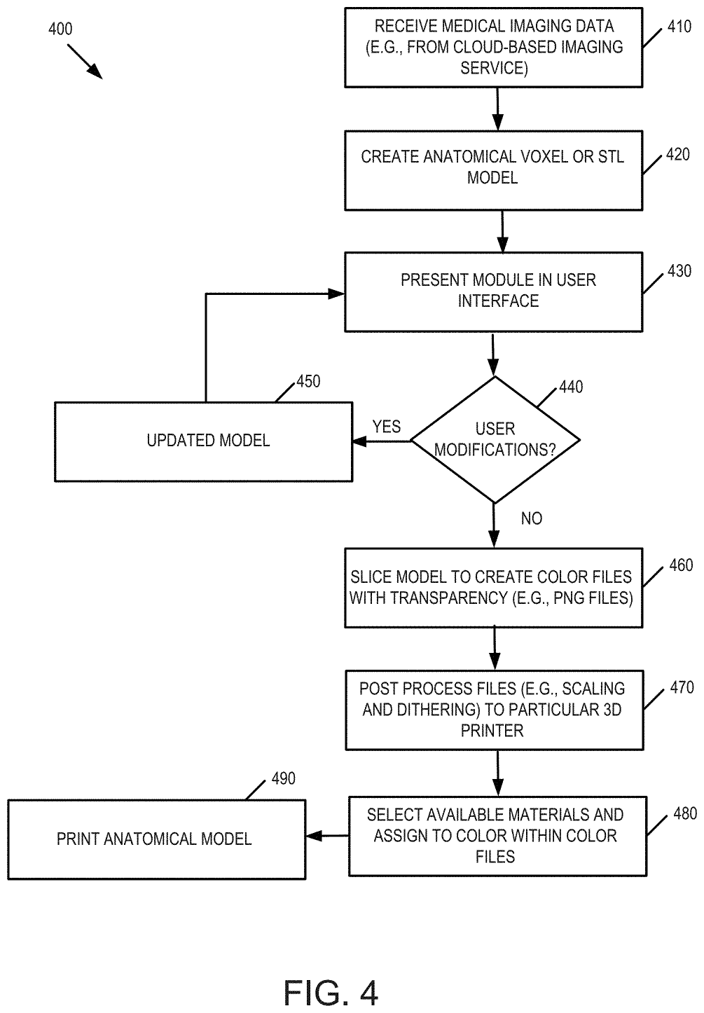

FIG. 4 is a flowchart illustrating a set of operations 400 for generating an anatomical structure according to one or more embodiments of the present technology. As illustrated in FIG. 4, receiving operation 410 receives imaging data (e.g., medical images) and/or computational structural analysis data. In accordance with various embodiments, this data may be stored on a cloud-based imaging service or locally on a computing device of a user. Modeling operation 420 creates a voxel or STL model of the structure (e.g., anatomical structure such as the heart) captured in the imaging and/or analysis data.

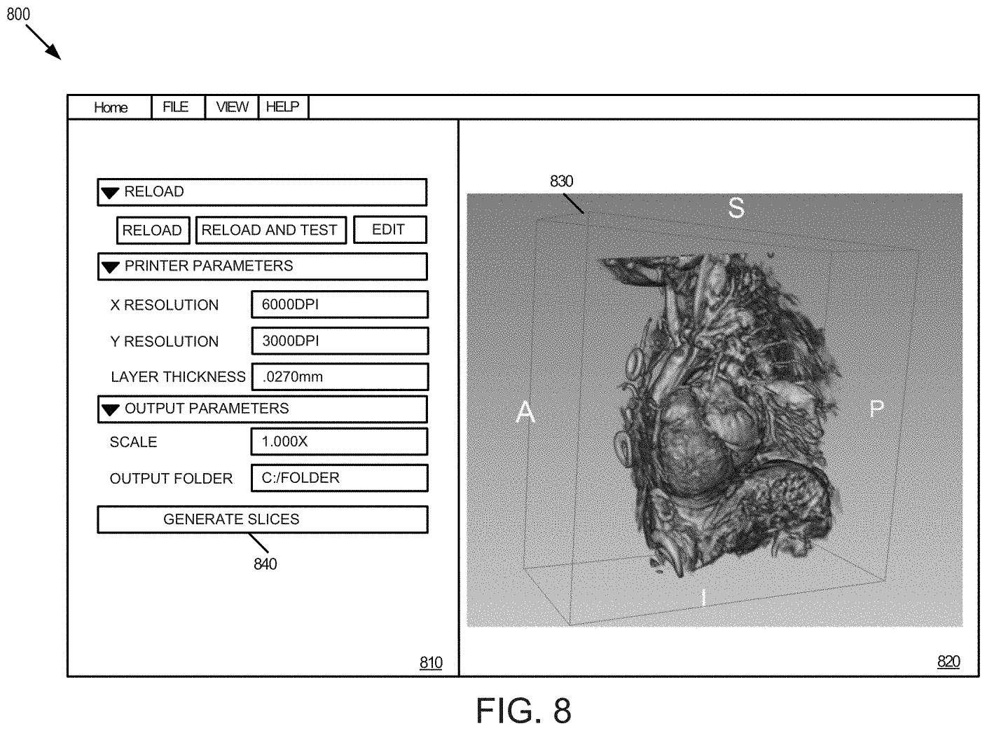

Once modeling operation 420 is finished, presentation operation 430 can present the model to the user via a user interface (see, e.g., graphical user interface 800 illustrated in FIG. 8). Determination operation 440 monitors interactions with the user interface to identify any user modifications (e.g., selections of areas of interest, modifications, color changes, and the like). When determination operation 440 identifies a user modification, the model can be updated using update operation 450 and presentation operation updates the model presented in the user interface. In some embodiments, the user may be able to access the underlying images and/or computation data via the user interface. For example, an additional widow pane can be presented with visualization of this the images or data allowing the user to compare the 3D model with the underlying data.

When determination operation 440 receives an indication that no more user modifications will be made the model can be finalized and slicing operation 460 applied. During slicing operation 460, the model can be sliced into multiple layer (e.g., 300 to 3000 layers) to create color files. These color files can be post-processed (e.g., scaled and dithered) to a particular 3D printer in post-processing operation 470. Availability operation 480 identifies the available materials from the printer and assigns a color within the color files to each of the materials. Then, printing operation 490 can print the 3D physical model (e.g., the anatomical model of a heart) captured by the original imaging and/or computational data.

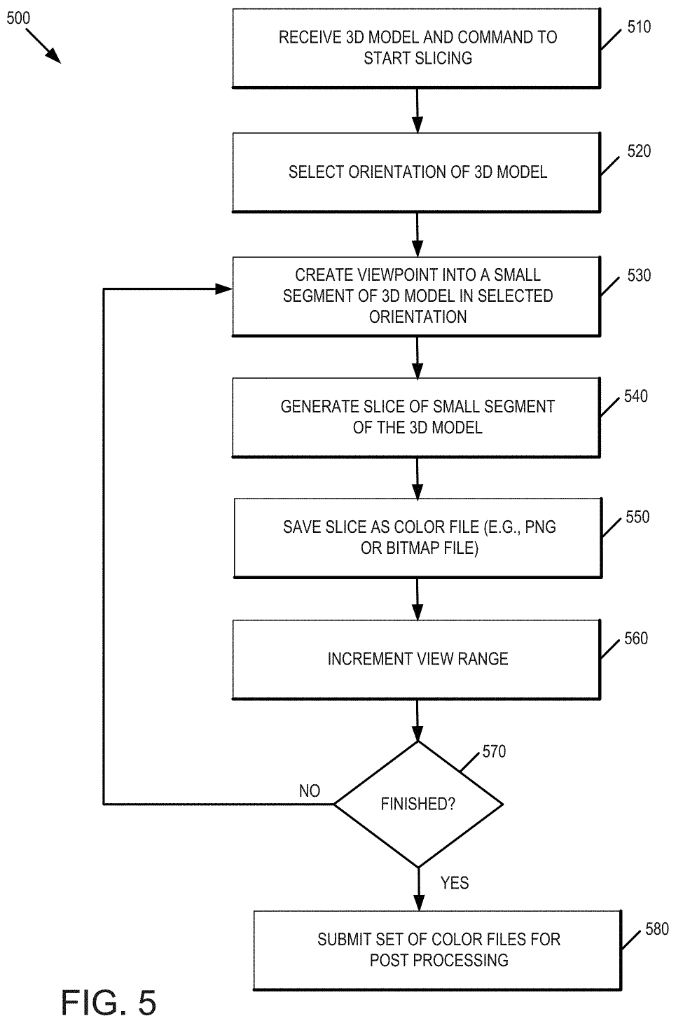

FIG. 5 is a flowchart illustrating a set of operations 500 for slicing a model in accordance with some embodiments of the present technology. During receiving operation 510 the 3D model and command to start slicing are received. Selection operation 520 selects an orientation of the 3D model. For example, in some embodiments the user can specify in a GUI which orientation to print. The selection of the orientation will affect the print time optimization and structural support during printing. For example, depending on the structure, it may be more efficient to print the structure on one side or the other because height equals increased print time. Segmentation operation 530 segments a small portion of the 3D model in the selected orientation to create a viewpoint for slicing with sling operation 540.

Recording operation 550 saves the slice as a color file. The color file may be a file type with transparency. During incrementation operation 560 the view range is incremented. Determination operation 570 determines if the entire 3D model has been sliced. When determination operation 570 determines that the entire model has not been sliced, then determination operation branches to creation operation 530 where segmentation operation 530 selects the next small portion of the 3D model in the selected orientation to create a viewpoint for slicing with slicing operation 540.

Various embodiments of the present technology use a color system of red, green, and blue (RGB) between 0-255 and an Alpha Channel for transparency in generating the PNG slices. The alpha channel is a color component that represents the degree of transparency (or opacity) of a color (i.e., the red, green and blue channels). It is used to determine how a pixel is rendered when blended with another. The system may be configured to create different slices for specific printers and based on the printer properties. For example, for a Stratasys j750 printer slices may be selected every 0.27 mm for proper mixing of materials to form a gradient. As a result, the slice thickness and distance between slices is a formula that is dependent on the machine. As a result, the following assignment can be set:

Slice thickness=X millimeters

Slice spacing=Y millimeters

Material 1=XXX-XXX-XXX RGB Value

Material 2=XXX-XXX-XXX RGB Value

Material 3=XXX-XXX-XXX RGB Value

Material 4=XXX-XXX-XXX RGB Value

Material 5=XXX-XXX-XXX RGB Value

Material 6=XXX-XXX-XXX RGB Value

When determination operation 570 determines that the entire model has been sliced, then determination operation branches to post-processing operation 580 where the set of color files are submitted for post processing.

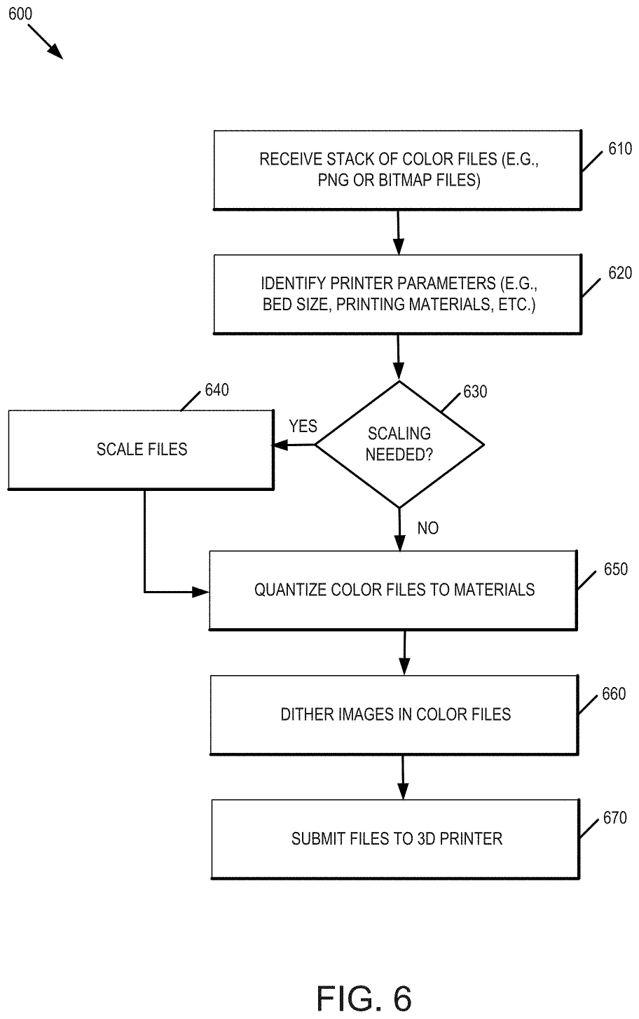

FIG. 6 is flowchart illustrating a set of operations 600 for post-processing sliced files in accordance with one or more embodiments of the present technology. During receiving operation 610 a stack of color files (e.g., PNG or BITMAP files) representing a sliced model of a structure are received. Identification operation 620 can identify printing parameters for the 3D printer. Examples of printer parameters include, but are not limited to bed dimensions, available printing materials, x-resolution, y-resolution, and z-resolution. This can be done, in accordance with various embodiments, by querying the printer, accessing a parameter file, or the like.

Determination operation 630 can determine whether scaling is needed. When determination operation 630 determines that scaling is needed, then determination operation 630 branches to scaling operation 640 to scale the images within the files. When determination operation 630 determines that scaling is not needed, then determination operation 630 branches to quantize color files to the available materials with quantization operation 650. Dithering operation 660 dither the images. In accordance with various embodiments, dithering can happen at 2 places. For example, some embodiments can dither the model prior to slicing. This is done through numerous 3D dithering algorithms including but not limited to the following: Random Dither, Halftone, and Error Diffusion. Dithering controls the blending behaviors of the materials allowing for the control of the characteristics of material-material interface. In some embodiments, dithering can also happen at the image level once the model has been sliced. 2D dithering algorithms can be applied over the image.

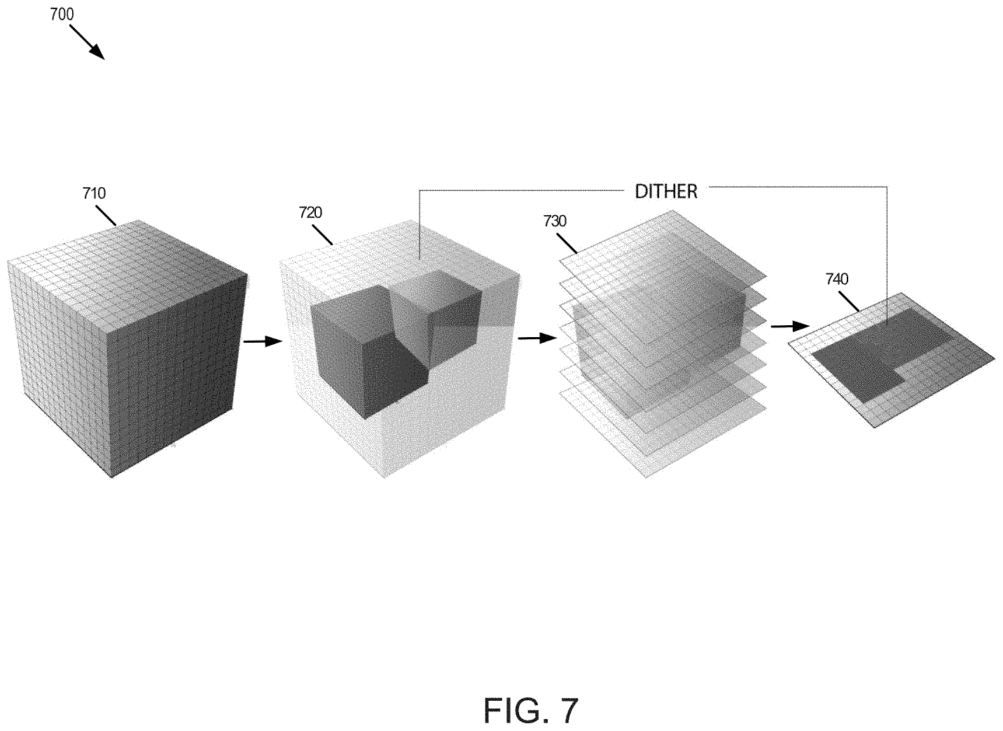

FIG. 7 is a flowchart illustrating a set of operations 700 for generating a printable file by establishing a voxel grid and dithering the model in accordance with some embodiments of the present technology. As illustrated in FIG. 7, voxel grid 710 is established providing a volumetric grid. Color and material channels are activated to represent the geometry of the structure 720 within the model within the voxel grid. The voxel model is then sliced horizontally into a set of 2D images 730 and then each pixel within the 2D image is assigned a color representing a material (or combination of materials) creating a printable file 740.

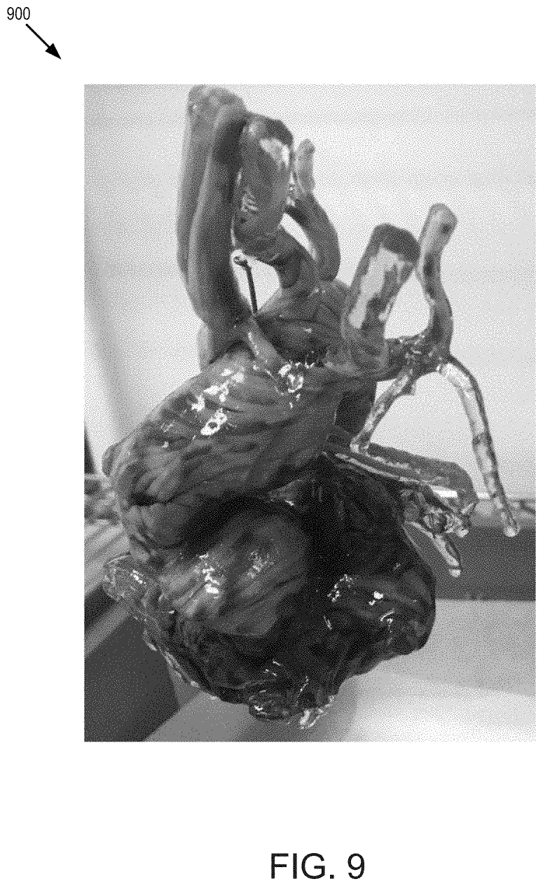

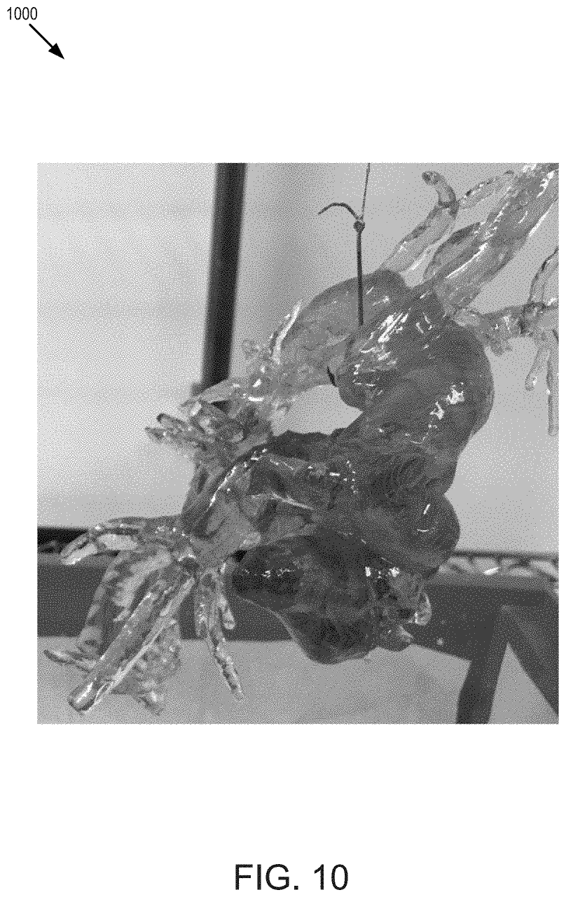

FIG. 8 is an example of a graphical user interface 800 that may be used according to various embodiments of the present technology. In the embodiments illustrated in FIG. 8, the user interface can include visualization pane 810 and navigational pane 820. Visualization pane 810 can be used to render the 3D CAD model of the structure (e.g., heart 830) loaded using navigational pane 820. The 3D CAD model can be rotated, enlarged, shrunk, and/or otherwise modified. For example, in some embodiments, the user may be able to hide, separate, or emphasize particular substructures, components, or layers within the 3D CAD model. Once selected, the user can then select button 840 to generate the slices which can be sent to the 3D printer for physical printing. FIG. 9 illustrates an example of a 3D printed heart 900 printed via the voxel-based 3D printing techniques according to one or more embodiments of the present technology. FIG. 10 illustrates an example of a selected portion of a heart 1000 printed via the voxel-based 3D printing techniques in accordance with various embodiments of the present technology.

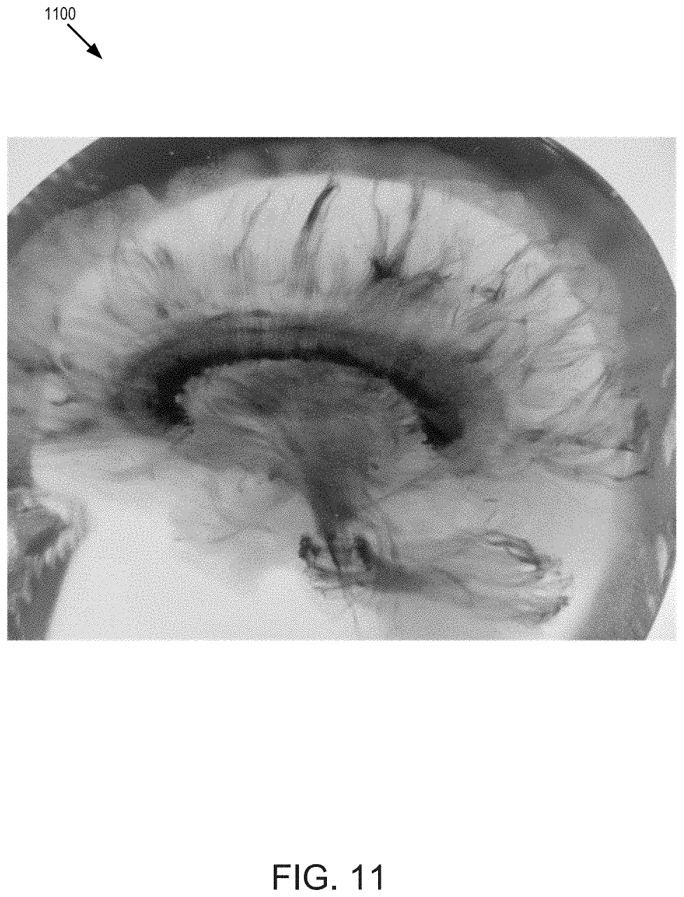

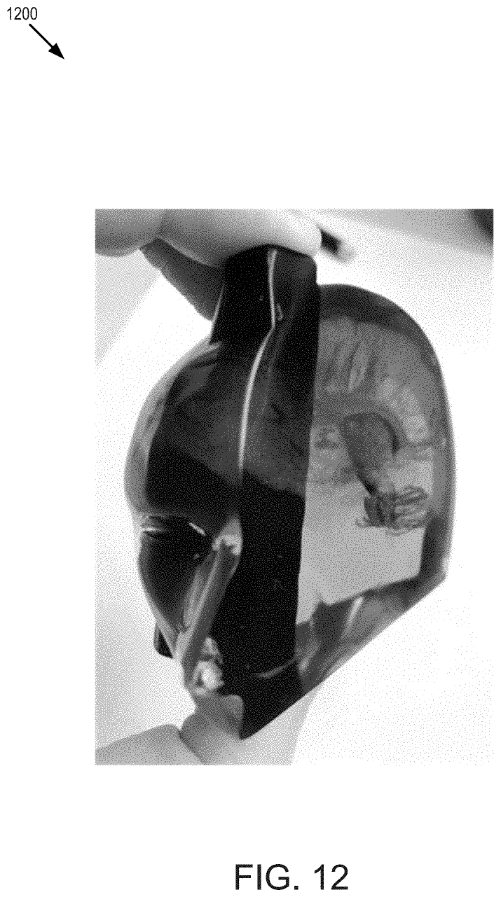

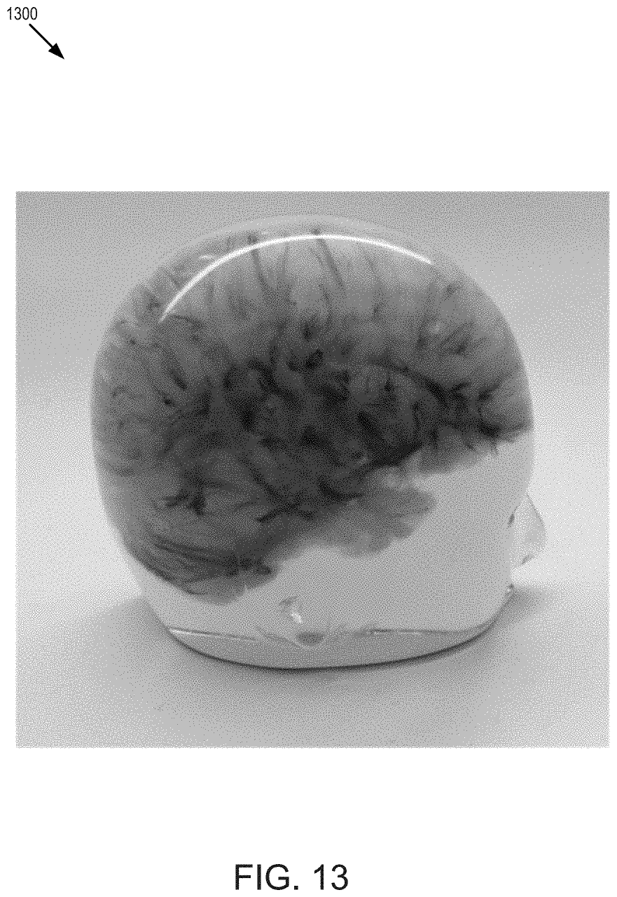

FIGS. 11-13 illustrate examples of a brain printed via the voxel-based 3D printing techniques in accordance with various embodiments of the present technology. In accordance with various embodiments, the printed structures can be printed in color and durometer and a combination of the two. Some parts of the structure may be printed with harder material while some parts are softer allowing a person interfacing with the structure to manipulate one or more parts of the 3D printed structure. In some embodiments, portions may be clear while other portions of the structure are printed in color to specifically demonstrate the anatomy.

### Exemplary Computer System Overview

Aspects and implementations of the analysis and printing system of the disclosure have been described in the general context of various steps and operations. A variety of these steps and operations may be performed by hardware components or may be embodied in computer-executable instructions, which may be used to cause a general-purpose or special-purpose processor (e.g., in a computer, server, or other computing device) programmed with the instructions to perform the steps or operations. For example, the steps or operations may be performed by a combination of hardware, software, and/or firmware.

FIG. 14 is a block diagram illustrating an example machine representing the computer systemization of the analysis and printing system. The analysis and printing system controller 1400 may be in communication with entities including one or more users 1425 client/terminal devices 1420, user input devices 1405, peripheral devices 1410, an optional co-processor device(s) (e.g., cryptographic processor devices) 1415, and networks 1430. Users may engage with the controller 1400 via terminal devices 1420 over networks 1430.

Computers may employ central processing unit (CPU) or processor to process information. Processors may include programmable general-purpose or special-purpose microprocessors, programmable controllers, application-specific integrated circuits (ASICs), programmable logic devices (PLDs), embedded components, combination of such devices and the like. Processors execute program components in response to user and/or system-generated requests. One or more of these components may be implemented in software, hardware or both hardware and software. Processors pass instructions (e.g., operational and data instructions) to enable various operations.

The controller 1400 may include clock 1465, CPU 1470, memory such as read only memory (ROM) 1485 and random access memory (RAM) 1480 and co-processor 1475 among others. These controller components may be connected to a system bus 1460, and through the system bus 1460 to an interface bus 1435. Further, user input devices 1405, peripheral devices 1410, co-processor devices 1415, and the like, may be connected through the interface bus 1435 to the system bus 1460. The interface bus 1435 may be connected to a number of interface adapters such as processor interface 1440, input output interfaces (I/O) 1445, network interfaces 1450, storage interfaces 1455, and the like.

Processor interface 1440 may facilitate communication between co-processor devices 1415 and co-processor 1475. In one implementation, processor interface 1440 may expedite encryption and decryption of requests or data. Input output interfaces (I/O) 1445 facilitate communication between user input devices 1405, peripheral devices 1410, co-processor devices 1415, and/or the like and components of the controller 1400 using protocols such as those for handling audio, data, video interface, wireless transceivers, or the like (e.g., Bluetooth, IEEE 1394a-b, serial, universal serial bus (USB), Digital Visual Interface (DVI), 802.11a/b/g/n/x, cellular, etc.). Network interfaces 1450 may be in communication with the network 1430. Through the network 1430, the controller 1400 may be accessible to remote terminal devices 1420. Network interfaces 1450 may use various wired and wireless connection protocols such as, direct connect, Ethernet, wireless connection such as IEEE 802.11a-x, and the like.

Examples of network 1430 include the Internet, Local Area Network (LAN), Metropolitan Area Network (MAN), a Wide Area Network (WAN), wireless network (e.g., using Wireless Application Protocol WAP), a secured custom connection, and the like. The network interfaces 1450 can include a firewall which can, in some aspects, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including, for example, the access and operation rights of an object by an individual, a machine, and/or an application, and the circumstances under which the permission rights stand. Other network security functions performed or included in the functions of the firewall, can be, for example, but are not limited to, intrusion-prevention, intrusion detection, next-generation firewall, personal firewall, etc., without deviating from the novel art of this disclosure.

Storage interfaces 1455 may be in communication with a number of storage devices such as, storage devices 1490, removable disc devices, and the like. The storage interfaces 1455 may use various connection protocols such as Serial Advanced Technology Attachment (SATA), IEEE 1394, Ethernet, Universal Serial Bus (USB), and the like.

User input devices 1405 and peripheral devices 1410 may be connected to I/O interface 1445 and potentially other interfaces, buses and/or components. User input devices 1405 may include card readers, finger print readers, joysticks, keyboards, microphones, mouse, remote controls, retina readers, touch screens, sensors, and/or the like. Peripheral devices 1410 may include antenna, audio devices (e.g., microphone, speakers, etc.), cameras, external processors, communication devices, radio frequency identifiers (RFIDs), scanners, printers, storage devices, transceivers, and/or the like. Co-processor devices 1415 may be connected to the controller 1400 through interface bus 1435, and may include microcontrollers, processors, interfaces or other devices.

Computer executable instructions and data may be stored in memory (e.g., registers, cache memory, random access memory, flash, etc.) which is accessible by processors. These stored instruction codes (e.g., programs) may engage the processor components, motherboard and/or other system components to perform desired operations. The controller 1400 may employ various forms of memory including on-chip CPU memory (e.g., registers), RAM 1480, ROM 1485, and storage devices 1490. Storage devices 1490 may employ any number of tangible, non-transitory storage devices or systems such as fixed or removable magnetic disk drive, an optical drive, solid state memory devices and other processor-readable storage media. Computer-executable instructions stored in the memory may include the imaging service 170 having one or more program modules such as routines, programs, objects, components, data structures, and so on that perform particular tasks or implement particular abstract data types. For example, the memory may contain operating system (OS) component 1495, modules and other components, database tables, and the like. These modules/components may be stored and accessed from the storage devices, including from external storage devices accessible through an interface bus.

The database components can store programs executed by the processor to process the stored data. The database components may be implemented in the form of a database that is relational, scalable and secure. Examples of such database include DB2, MySQL, Oracle, Sybase, and the like. Alternatively, the database may be implemented using various standard data-structures, such as an array, hash, list, stack, structured text file (e.g., XML), table, and/or the like. Such data-structures may be stored in memory and/or in structured files.

The controller 1400 may be implemented in distributed computing environments, where tasks or modules are performed by remote processing devices, which are linked through a communications network, such as a Local Area Network (“LAN”), Wide Area Network (“WAN”), the Internet, and the like. In a distributed computing environment, program modules or subroutines may be located in both local and remote memory storage devices. Distributed computing may be employed to load balance and/or aggregate resources for processing. Alternatively, aspects of the controller 1400 may be distributed electronically over the Internet or over other networks (including wireless networks). Those skilled in the relevant art(s) will recognize that portions of the analysis and printing system may reside on a server computer, while corresponding portions reside on a client computer. Data structures and transmission of data particular to aspects of the controller 1400 are also encompassed within the scope of the disclosure.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a computer-readable medium claim, other aspects may likewise be embodied as a computer-readable medium claim, or in other forms, such as being embodied in a means-plus-function claim. Any claims intended to be treated under 35 U.S.C. § 112(f) will begin with the words “means for”, but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. A method of three-dimensional volumetric printing comprising:
receiving medical imaging data regarding an anatomical structure;
creating, from the medical imaging data, a computer-aided design (CAD) model of the anatomical structure;
slicing the CAD model into multiple layers to create a set of color files in a format supporting transparency; and
generating, based on the set of color files representing the multiple layers of the CAD model, instructions configured to control a three-dimensional (3D) printer supporting multiple materials to produce a 3D model of the anatomical structure using the multiple materials.

2. The method of claim 1, wherein slicing the CAD model into multiple layers to create the set of color files comprises:
selecting an orientation of the CAD model;
generating a viewpoint into a segment of the CAD model in the selected orientation; and
until all segments of the CAD model have been captured:
taking a snapshot of the segment; and
saving the snapshot as a color file.

3. The method of claim 1, further comprising post-processing the set of color files based on printing parameters of the 3D printer.

4. The method of claim 3, wherein the post-processing of the set of color files includes:
automatically identifying the printer parameters by querying the 3D printer,
wherein the printer parameters include the dimensions of a printing bed, x-resolution, and y-resolution; and
scaling the multiple layers within the set of color files to allow the 3D model of the anatomical structure produced by the 3D printer to fit on the dimensions of the printing bed.

5. The method of claim 3, wherein the post-processing of the set of color files includes:
quantizing the multiple layers within the set of color files to correspond to the multiple materials available for printing via the 3D printer; and
dithering the multiple layers within the set of color files.

6. The method of claim 1, wherein the 3D model of the anatomical structure produced by the 3D printer includes at least one layer with a gradient with two or more of the multiple materials.

7. The method of claim 1, wherein the CAD model includes a voxel-based model or a stereolithography (STL) model.

8. The method of claim 1, wherein the format supporting transparency includes a GIF file format, a PNG file format, a BMP file format, a TIFF file format, or a JPEG 2000 file format.

9. The method of claim 1, wherein the medical imaging data includes radiographic images or four-dimensional (4D) flow representing blood flow and velocity.

10. The method of claim 1, wherein the medical imaging data regarding the anatomical structure is retrieved from a cloud-based imaging platform.

11. A system comprising:
a processor;
a database having stored thereon multiple digital files representing a structure;
an three-dimensional modeling module, under control of the processor, configured to—
process the multiple digital files; and
create three-dimensional (3D) model of the structure from the multiple digital files; and,
a slicing module, under control of the processor, configured to create multiple color files, wherein each of the multiple color files represent a layer of the structure, and wherein the multiple color files support transparency to allow removal of one or more components of the structure without additional processing; and
a printing module, under control of the processor, configured to generate, based on the multiple color files representing the multiple layers of the structure, instructions configured to control a 3D printer supporting multiple materials to produce a physical 3D model of the structure using the multiple materials.

12. The system of claim 11, wherein the slicing module creates the multiple color files by:
selecting an orientation of the 3D model;
generating a viewpoint into a segment of the 3D model in the selected orientation; and
until all segments of the 3D model have been captured:
taking a snapshot of the segment of the 3D model of the structure; and
saving the snapshot as a color file.

13. The system of claim 11, further comprising a user interface module configured to generate a graphical user interface to be presented on a display, wherein the graphical user interface includes controls to allow a user to select which of the one or more components of the structure to print.

14. The system of claim 11, wherein the digital files include a finite element analysis of the structure representing stresses within the structure and the stresses are represented in the physical 3D model printed by the 3D printing using combinations of the materials.

15. The system of claim 11, wherein the combinations of the materials are combined to create gradient of materials over a portion of the physical 3D model.

16. The system of claim 11, further comprising a post-processing module, under control of the processor, configured to—
automatically identify printer parameters by querying the 3D printer,
wherein the printer parameters include the dimensions of a printing bed, x-resolution, and y-resolution; and
scale the layer of the structure within each of the multiple color files to allow the physical 3D model of the structure produced by the 3D printer to fit on the dimensions of the printing bed.

17. A non-transitory computer-readable medium storing having instructions stored thereon that when executed by one or more processors cause a machine to:
receive data regarding a structure, wherein the data includes one or more images, four-dimensional flows, finite element analyzes, or topological optimizations of the structure;
create, from the data, a three-dimensional (3D) model of the structure having multiple components,
wherein the 3D model includes a voxel-based model or a stereolithography (STL) model;
slicing the 3D model of the structure into multiple layers to create a set of color files in a format supporting transparency to allow for removal of one or more of the multiple components without additional processing; and
generate, based on the set of color files representing the multiple layers of the 3D model of the structure, instructions configured to control a 3D printer supporting multiple materials to produce a physical model of the structure using the multiple materials.

18. The non-transitory computer-readable medium of claim 17, wherein the instructions when executed by the one or more processors slice the 3D model into multiple layers to create the set of color files by causing the machine to:
select an orientation of the 3D model;
generate a viewpoint into a segment of the 3D model in the selected orientation; and
until all segments of the 3D model have been captured:
taking a snapshot of the segment of the structure; and
saving the snapshot as a color file.

19. The non-transitory computer-readable medium of claim 17, wherein the instructions when executed by the one or more processors post-process the set of color files based on printing parameters of the 3D printer by causing the machine to:
identify the printer parameters including the dimensions of a printing bed, x-resolution, and y-resolution;
scale the multiple layers within the set of color files to allow the 3D model of the structure produced by the 3D printer to fit on the dimensions of the printing bed;
quantize the multiple layers within the set of color files to correspond to the multiple materials available for printing via the 3D printer; and
dither the multiple layers within the set of color files.

20. The non-transitory computer-readable medium of claim 17, wherein the structure includes an anatomical structure.
